(19) United States
(12) Patent Application Publication
(10) Pub. No.: US 2014/0012046 A1
(43) Pub. Date: Jan. 9, 2014

(54) ISOMERISATION CATALYST

(71) Applicants: Lutz Heuer, Dormagen (DE); Eberhard Zirngiebl, Koln (DE); Martin Mechelhoff, Koln (DE)

(72) Inventors: Lutz Heuer, Dormagen (DE); Eberhard Zirngiebl, Koln (DE); Martin Mechelhoff, Koln (DE)

(73) Assignee: LANXESS DEUTSCHLAND GMBH, Leverkusen (DE)

(21) Appl. No.: 13/811,324

(22) PCT Filed: Jul. 22, 2011
(86) PCT No.: PCT/EP2011/062649

§ 371 Date: Sep. 11, 2013

(30) Foreign Application Priority Data
Jul. 23, 2010 (EP) 10007661.1

(51) Int. Cl. C07C 29/56; B01J 31/02

(52) U.S. Cl. CPC C07C 29/56; B01J 31/0204
USPC 568/829; 502/171

(57) ABSTRACT

The present invention relates to a process for preparing menthol isomers by selective rearrangement of stereoisomers of menthol or mixtures thereof in the presence of ruthenium-containing supported catalysts doped with or comprising alkaline earth metal alkoxides, and to the catalysts themselves.

The present invention relates to a process for preparing menthol isomers by selective rearrangement of stereoisomers of menthol or mixtures thereof in the presence of ruthenium-containing supported catalysts doped with or comprising alkaline earth metal alkoxides, and to the catalysts themselves.

Among the naturally occurring cyclic terpene alcohols, l-menthol, the main constituent of peppermint oil, takes a special place due to its cooling and refreshing effect. l-Menthol therefore finds use as an odourant or flavouring and is used in the pharmaceutical industry.

Menthol preparation by catalytic hydrogenation of compounds having the carbon skeleton of menthane with at least one double bond and having oxygen substitution in the 3 position, for example thymol, leads to a stereomer mixture of the eight optically active menthols: d,l-menthol, d,l-isomenthol, d,l-neomenthol and d,l-neoisomenthol. These eight optically active menthols differ in relation to their organoleptic properties. Only l-menthol has the characteristic peppermint odour and the refreshing effect already mentioned. It is therefore the most economically important of the menthol stereoisomers. Other isomers of menthol have other organoleptic properties which are desirable for other uses. It is therefore a general aim to conduct the hydrogenation, through suitable choice of the reaction conditions and of the catalysts, in such a way that a maximum amount of d,l-menthol is formed. By separating the mixture of the stereoisomeric menthols, d,l-menthol is then obtained as a racemate, which can then be split into the antipodes by means of methods known per se. Once they have been split, stereoisomers of menthol, in the presence of a selective catalyst, offer the possibility of obtaining other isomers of menthol, likewise as optically enriched or pure antipodes.

The boiling points of d,l-isomenthol (218.6° C. at 1013 hPa; 75 to 78° C. at 3.3 hPa) and d,l-menthol (216.5° C. at 1013 hPa; 75 to 78° C. at 3.3 hPa) are very close to one another. The separating performance of a column in the distillative separation of the individual menthol isomers is therefore determined particularly by the ratio of d,l-menthol to d,l-isomenthol. A high space-time yield of d,l-menthol in the distillative separation therefore necessitates not only a maximum d,l-menthol content in the mixture to be separated, but also a minimum d,l-isomenthol content. The yield of menthol for a given distillation column is thus determined essentially by the input ratio of d,l-menthol to d,l-isomenthol.

For preparation of d,l-menthol, it is known that compounds having the carbon skeleton of menthane with at least one double bond and having oxygen substitution in the 3 position, for example thymol, in continuous processes can be hydrogenated with hydrogen over fixed catalyst beds, or stereoisomers of menthol can be rearranged over fixed catalyst beds.

DE 23 14 813 A1 describes a process for hydrogenating compounds having the carbon skeleton of menthane with at least one double bond and having oxygen substitution in the 3 position over a bed of a cobalt-manganese catalyst at temperatures of 170° C. to 220° C. and a pressure exceeding 25 bar, preferably exceeding 200 bar. In the examples, temperatures of 180° C. to 210° C. and pressures exceeding 200 bar are employed, and a mixture of the eight stereoisomeric menthols is obtained, which consists to an extent of 59.5 to 59.9% of racemic d,l-menthol and to an extent of 10.6 to 10.8% of d,l-isomenthol. The maximum menthol/isomenthol ratio is 5.7. Modification of the cobalt-manganese catalyst with copper affords menthol mixtures with d,l-menthol contents of 57.6% and d,l-isomenthol contents of 9.2%, which corresponds to a menthol/isomenthol ratio of about 6.3. The resulting mixtures, however, have 4 to 5% of undesirable by-products in the form of non-reutilizable hydrocarbons.

EP 0 563 611 A 1 and DE 197 18 116 A 1 disclose that the hydrogenation of aromatic or partly hydrogenated cyclic compounds having the carbon skeleton of menthane with at least one C═C double bond and having oxygen substitution in the 3 position can be performed with hydrogen over a fixed bed catalyst comprising palladium, ruthenium or rhodium or a mixture of these elements as active constituents and alkali metal hydroxides and/or sulphates as promoters, in each case applied to a support, the support being doped with a metal from the rare earths and manganese. In the examples, temperatures of 180 to 240° C. and pressures of 270 to 300 bar were employed. This afforded menthol mixtures containing approx. 52 to 57% d,l-menthol and 11.5 to 14.8% d,l-isomenthol, which corresponds to a menthol/isomenthol ratio of 3.6 to 4.4.

EP 743 296 A 1 discloses catalysts which consist of support-free, compressed powders of cobalt oxides or hydroxides, manganese oxides or hydroxides and alkaline earth metal oxides or hydroxides, and are used at temperatures of 150° C. to 230° C. and pressures of 25 to 350 bar. In the examples cited, temperatures exceeding 165° C. and pressures of more than 200 bar are employed. The composition of the menthol mixtures formed is not discussed.

The rearrangement of stereoisomers of l-menthol is described in U.S. Pat. No. 5,756,864: at temperatures of 200 to 350° C. and hydrogen pressures of 50 to 350 bar, preferably 100 to 300 bar, d-menthol is racemized and isomerized over a catalyst in a continuous process, the catalyst consisting of support-free, compressed powders of nickel oxides or hydroxides, manganese oxides or hydroxides and alkaline earth metal oxides or hydroxides. This afforded menthol mixtures which consisted to a maximum extent of 59.8% of d,l-menthol.

U.S. Pat. No. 2,843,636 discloses performing the isomerization of stereoisomers of menthol to d,l-menthol with hydrogen in the presence of a hydrogenation catalyst from the group of copper chromite, cobalt and nickel at 260 to 280° C. and 500 to 1300 p.s.i.g. (34 to 90 bar) in autoclaves. The resulting mixtures had, as well as approx. 10 to 12% d,l-isomenthol, a d,l-menthol content of 60 to 64%.

DE 198 53 562 A describes a low-pressure hydrogenation of thymol over a stationary catalyst bed having a temperature gradient: the first two of five series-connected reactor tubes are heated to 180° C., the three downstream reactor tubes to 80 to 90° C. With a catalyst comprising, on a support doped with a metal from the rare earths and with manganese, ruthenium as the active constituent and alkali metal hydroxides as promoters, it was possible at a pressure of 3 bar to obtain a menthol isomer mixture which contained 64.4% by weight of menthol and 12.1% isomenthol, which corresponds to a menthol/isomenthol ratio of 5.3. The isomerization of a hydrogen-saturated mixture of d,l-neomenthol, d,l-isomenthol and d,l-menthol at standard pressure afforded an isomer mixture with a composition of 65.3% d,l-menthol and 12.1% isomenthol. In this low-pressure process, it is possible to achieve high menthol contents of approx. 65%. The maximum menthol/isomenthol ratio, however, is 5.4.

DE 100 23 283 A describes an improved process in which isomer mixtures having typically about 55% d,l-menthol, by isomerization with simple supported ruthenium catalysts, can prepare menthol-richer mixtures having up to 67.3% d,l-menthol and only 8.2% d,l-isomenthol i.e. a menthol/isomenthol ratio of up to 8.1. In addition, DE 100 23 283 A discloses that the catalysts can be regenerated with alkoxides, oxides and hydroxides of the alkali metals or alkaline earth metals.

A feature common to all known process is accordingly that they give at least 8.2% d,l-isomenthol and allow maximum menthol/isomenthol ratios of 8.1.

It was therefore an object of the invention to find a selective and technically simple process for the preparation of d,l-menthol which gives only small amounts of d,l-isomenthol and allows high menthol/isomenthol ratios, with simultaneous substantial avoidance of the formation of unwanted by-products.

In a further aspect, it was an object of the invention to configure this conversion of essentially pure stereoisomers of menthol with maximum selectivity, such that they can be converted catalytically at least partly to another stereoisomer, without losing the optical activity.

First of all, it should be noted that the percentages stated hereinafter, as is also customary in the prior art, are understood to mean area per cent which are obtained in the gas chromatography analysis of the product mixture. Menthol/isomenthol ratio is consequently understood to mean the ratio of area per cent (GC) of d,l-menthol to area per cent (GC) of d,l-isomenthol.

A catalyst comprising ruthenium applied to a support material has now been found, wherein the support material is aluminium oxide and the catalyst is characterized in that
it comprises at least one alkaline earth metal alkoxylate or
is obtainable by reacting a catalyst comprising ruthenium applied to a support material, the support material being aluminium oxide, with at least one alkaline earth metal alkoxylate.

The invention further provides a process for isomerizing stereoisomers of menthol or mixtures of such stereoisomers in the presence of the aforementioned catalyst.

The subject-matter of the invention includes not just the general or preferred embodiments disclosed for individual parameters or compounds, but also every conceivable combination thereof.

The aluminium oxide used as the support material can be used in all known polymorphs, preferably in the γ polymorph. Advantageously, the aluminium oxide used as the support material has a BET surface area of at least 100 m²/g, preferably at least 160 m²/g and more preferably at least 180 m²/g. Particular preference is given to aluminium oxide which additionally has a high proportion of macroporous pores having a pore diameter of at least 50 nm and a pore volume of at least 300 mm³/g, preferably at least 600 mm³/g. Examples of suitable support materials include the commercially available aluminium oxides SPH 1515, SPH 531, SPH 501 from Rhodia, D 10-10 from BASF and SA 6176 from Norton.

The support material can be used, for example, in the form of powder with particle sizes of 0.001 to 0.1 mm, crushed and sieved material with particle sizes between 0.05 and 5 mm or in shaped bodies such as extrudates, pills, balls or granules having diameters of 0.2 to 30 mm.

For example, the procedure for preparation of the catalysts may be to first apply ruthenium and optionally one or more further metals from transition group 8 of the periodic table and/or tin and/or zinc to one of the support materials mentioned. The application can be accomplished by treatment, for example impregnation or spraying, of the support material with solutions of salts of the metals. For this purpose, for example, the chlorides, acetates and/or nitrates are used. This application of the metals can be effected in one step with dissolved mixtures of the salts, or successively with the solutions of the individual compounds. After each application, the catalyst can be dried.

The amount of ruthenium or of ruthenium compound for preparation of the inventive catalyst is, or is selected such that the catalyst, based on and calculated on ruthenium, is, for example, 0.1 to 35% by weight, preferably 1 to 10% by weight.

A catalyst prepared in the manner stated is reduced, for example, by treatment with hydrogen or hydrogen-containing gases, for example nitrogen having a hydrogen content of 0.5 to 10% and preferably 0.5 to % by volume of hydrogen, for example at a temperature of 20 to 400° C., preferably 30 to 250° C. The reduction can also be effected with other reducing agents, for example hydrazine. The reduced catalyst is preferably washed thereafter in order to remove any salts still adhering.

The metal applied can, for example, also be fixed on the support by treating the support which has been impregnated with ruthenium and optionally further metals with a solution of basic salts, for example alkali metal or alkaline earth metal hydroxides or oxides, for example sodium hydroxide or potassium hydroxide, precipitating the metal as the oxide or hydroxide. If the metal has been fixed on the support, the reduction and the washing to remove soluble constituents can be conducted in any sequence. Preferably, the soluble constituents are first removed by washing and the catalyst is then reduced. The reduction can also be effected in situ in the course of performance of the process according to the invention.

After each washing step with metal salt solution or basic salts or after washing operations, a drying step can be conducted. However, it is also possible to use the support without drying in the next preparation step.

The catalyst thus prepared can then, in one embodiment, be doped with alkaline earth metal hydroxides, such as preferably barium hydroxide, for example at 20° C. up to the boiling point of the solution, the doping preferably being effected in such a way that the catalyst simultaneously comes into contact with the solution of alkaline earth metal hydroxides, such as preferably barium hydroxide. The water needed for this purpose is absorbed by the shaped catalyst bodies. The spraying can be effected under ambient conditions or under inert gas.

A catalyst thus prepared can, just like an undoped catalyst, be reduced in the reaction system and used for isomerization (example 1, contrastive example to DE 198 53 562 and DE 100 23 283).

According to the invention, a further increase in activity and selectivity is achieved when the catalyst which has already been doped with alkaline earth metals or a catalyst which has not been doped with alkaline earth metals is doped (further) by contacting with at least one alkaline earth metal alkoxylate.

For this purpose, it is possible to use, for example, alkaline earth metal alkoxylates of the formula (I)

(R—O)₂M (I)

in which
R in each case independently, preferably identically, is a primary, secondary or tertiary, cyclic or acyclic, branched or unbranched C1 to C20-alkyl radical which may optionally be further substituted by aryl, C₁-C₄-alkoxy or C₆ to C₁₄-aryloxy and is preferably methyl, ethyl, n-propyl, isopropyl, n-butyl, sec-butyl, tert-butyl, n-pentyl, neopentyl, n-hexyl, cyclohexyl or the stereoisomeric menthyl radicals and
M is calcium, strontium or barium, preferably barium.

The preferred barium alkoxylates can be obtained, for example, by reacting barium perchlorate with the corresponding potassium alkoxylates, preferably dissolved in the same or another alcohol, forming sparingly soluble potassium perchlorate, which can be removed easily from the reaction solutions, for example, by filtration.

Barium menthoxides are, for example, also obtainable by admixing barium ethoxide or barium isopropoxide with an excess of menthol stereoisomers and leaving the mixture to stand for a prolonged period or heating said mixture.

Particular preference is given to using barium ethoxide, 10% w/v in ethanol, barium isopropoxide as a solid substance, dissolved in menthol isomers, or barium isopropoxide, 20% w/v in isopropanol.

The doping can be effected, for example, by adding the alkaline earth metal alkoxylates, for example in solution of an alcohol other than menthol, to the feed stream of the menthols used as reactants, for example of the stereoisomer mixtures of the menthols, and doping the catalyst further or for the first time in this way.

This process has the advantage that, for example, catalysts doped with alkaline earth metal hydroxides are not exposed to the air, under which alkaline earth metal carbonates are formed with the carbon dioxide in the air.

The amount of the alkaline earth metal alkoxylate which is used for preparation of the inventive catalyst is, or is selected such that the molar ratio of ruthenium to alkaline earth metal is, for example, 30:1 to 1:30, preferably 10:1 to 1:10.

The inventive catalyst is especially suitable for preparation of d,l-menthol by catalytic isomerization of stereoisomers of menthol or mixtures of these stereoisomers, and additionally for stereoselective conversion of
(+)-menthol (d-menthol) to (−)-neomenthol,
(−)-menthol (l-menthol) to (+)-neomenthol,
(+)-isomenthol to (−)-neoisomenthol or
(−)-isomenthol to (+)-neoisomenthol.

Therefore, the invention also encompasses the use of the inventive catalysts for isomerization of stereoisomers of menthol or mixtures of these stereoisomers.

The reactant used may be either the essentially pure isomers of menthol or any desired mixtures of stereoisomers thereof.

Essentially pure stereoisomers mean a content of the respective stereoisomer of menthol, based on the total content of the 8 isomeric menthols, of 95% or more, preferably 98% or more, more preferably 99% or more.

Mixtures of stereoisomers are obtained, for example, in the racemization of optically active menthols or remain in the case of distillative removal of d,l-menthol from a stereoisomer mixture. It is also possible, for example, to use menthol isomer mixtures which form in the case of hydrogenation of cyclic compounds which have the carbon skeleton of menthane with at least one double bond and are substituted by oxygen in the 3 position, for example thymol, menthone or isomenthone.

Such starting mixtures contain, for example, between:
0 and 100%, preferably 30 to 70%, d,l-menthol or D- or L-menthol
0 and 100%, preferably 2 to 30%, d,l-isomenthol or (+)- or (−)-isomenthol
0 and 100%, preferably 10 to 99%, d,l-neomenthol or (+)- or (−)-neomenthol
0 and 100%, preferably 0.1 to 70%, d,l-neoisomenthol or (+)- or (−)-neoisomenthol,
with the proviso that at least two stereoisomers must be present in the mixture, the sum of the eight aforementioned compounds adds up to 90 to 100%, preferably to 95 to 100%, of the starting mixture, and the maximum content of any stereoisomer of menthol in the mixture, based on the total content of the 8 isomeric menthols, is below 95%.

Preferred mixtures are those which contain
30 to 70% d,l-menthol or D- or L-menthol
2 to 30% d,l-isomenthol or (+)- or (−)-isomenthol
10 to 99% d,l-neomenthol or (+)- or (−)-neomenthol and
0.1 to 70% d,l-neoisomenthol or (+)- or (−)-neoisomenthol,
with the proviso that the sum of the eight aforementioned compounds adds up to 90 to 100%, preferably to 95 to 100%, of the starting mixture.

Particularly preferred mixtures are those which contain
30 to 70% d,l-menthol
2 to 30% d,l-isomenthol
10 to 99% d,l-neomenthol and
0.1 to 70% d,l-neoisomenthol,
with the proviso that the sum of the four aforementioned racemic enantiomer pairs adds up to 90 to 100%, preferably to 95 to 100%, of the starting mixture.

The isomerization can be performed, for example, in reactors known per se and either batchwise or continuously, preference being given to continuous performance.

In batchwise processes, the catalyst hourly space velocity is, for example, 0.0001 to 10 kg of catalyst per kg of reactant, preferably 0.01 to 0.1 kg of catalyst per kg of reactant.

In continuous processes, the catalyst hourly space velocity is, for example, 0.005 to 5 kg of starting material per litre of catalyst and hour [kg/l*h], preferably 0.03 to 2 kg/l*h, more preferably 0.06 to 1.0 kg/l*h.

With increasing catalyst hourly space velocity, the space-time yield for the process according to the invention increases. At the same time, however, the menthol/isomenthol ratio falls, the degree of decrease depending strongly on the reaction temperature selected.

The maximum catalyst hourly space velocity for a given reaction temperature at which the desired result is still achieved is easy for the person skilled in the art to determine in a few tests.

The process according to the invention can be performed, for example, in a stirred tank, as a trickle phase, in the liquid phase with slurried catalyst, as a bubble column or over a stationary catalyst bed. Preference is given to performing the process according to the invention in the liquid phase in reactors with stationary catalyst beds.

The reactor may, for example, be connected downstream of a reactor for hydrogenation of cyclic compounds having the carbon skeleton of menthane with at least one double bond and having oxygen substitutes in the 3 position, or of a reactor for racemization/isomerization of d-menthol or other isomers of l-menthol, or of a separating apparatus such as, more particularly, a distillation or rectification plant for stereoisomer mixtures of menthol.

The reactor in which the process according to the invention is performed may, for example, also be connected between a reactor for hydrogenation, isomerization or racemization and a downstream separating apparatus such as, more particularly, a distillation or rectification plant.

It is optionally possible to use various product streams, for example from the hydrogenation and/or separation, in a mixture as the reactant in the process according to the invention. The reactor for isomerization may, however, optionally also be operated in isolation.

In one embodiment of the connection of such a reactor filled with the inventive catalyst, it is advantageous to use an already rectified menthol stream which is low in d,l-menthol as the reactant, very particular preference being given to using a neomenthol-containing stream.

The d,l-menthol-containing isomer mixture prepared by the process according to the invention can be separated in a manner known per se for isolation of pure d,l-menthol, for example by distillation or rectification.

The process of the invention can he performed in the presence of solvents. Preference is given, however, to a solvent-free procedure.

The process according to the invention can be performed, for example, at a total pressure of 25 hPa to 30 MPa, preferably 50 hPa to 5 MPa, more preferably at 100 hPa to 2 MPa and most preferably at 1000 hPa to 1 MPa.

The process according to the invention can also be conducted with addition or without addition of hydrogen:

In the case of addition of hydrogen, for example, the liquid phase is saturated with hydrogen prior to entry into the reactor, or gaseous hydrogen is passed into the reactor together with the reactant, such that a partial hydrogen pressure between 1 hPa and 30 MPa, preferably between 10 hPa and 5 MPa, more preferably between 10 hPa and 1 MPa, is established.

Preferably, however, no hydrogen is added in the course of performance of the process according to the invention, and so any hydrogen present is that which may be introduced into the reactor via the reactants used.

The process according to the invention is performed, for example, at temperatures of 30 to 170° C., preferably at temperatures of 50 to 150° C., more preferably at temperatures of 70 to 130° C. and more preferably 75 to 115° C.

The advantage of the present invention can be considered to be especially that the process according to the invention using the inventive catalyst allows the highly selective isomerization of stereoisomers of menthol, which allows the subsequent workup to be configured with very particular efficiency. When proceeding from pure, industrially available starting materials such as d-menthol or l-menthol or neomenthol, menthol/isomenthol ratios of more than 400 are achievable.

The examples which follow are intended to illustrate the invention, but without limiting the subject-matter thereof thereto.

### EXAMPLES

### Example 1

### Preparation of the Catalyst

1000 g (approx. 2.24 l) of a commercial γ-Al₂O₃ with a BET surface area of approx. 255 m²/g (SA 6176 from Norton, extrudate with particle diameter 1/16″ (approx. 1.6 mm), bulk density approx. 0.44 kg/l) was initially charged in a large rotary evaporator (10 l flask), an aqueous solution of 300 g of ruthenium (III) chloride (purchasable solution, 20% by weight of Ru, 60 g of Ru) in 1153 g of distilled water was added, and the mixture was rotated for 10 minutes (approx. 16 rpm (revolutions per minute)). The solvent was distilled off at 90° C. and 10 mbar. The catalyst was reduced in a hydrogen stream at 250° C. Subsequently, the catalyst was washed with distilled water until the wash water was chloride-free. Thereafter, the catalyst was dried in the rotary evaporator (90° C., 10 mbar).

2000 ml (bulk density approx. 0.5 kg/l) of the catalyst thus obtained were then sprayed with a solution of 50 g of barium hydroxide octahydrate in 150 ml of distilled water at 75 to 90° C., such that a homogeneous white precipitate formed on the previously black shaped catalyst bodies. In the course of this, the catalyst warmed up slightly.

This catalyst was used for comparison under the conditions specified in Table 1.

### Example 2

### Doping

The catalyst according to Example 1 was then doped with barium ethoxylate solution, 80 ml, 10% by weight in ethanol, introduced into 500 ml of menthol isomers as the feed stream during use. A highly active catalyst is thus obtained, which exhibits the improved results shown in Tables 2, 3, 4 and 5.

### Example 3

### Test Plant

The test plant consisted of thermostated reactor tubes each of length 1 m and internal diameter 45 mm. The reactors were heated by means of thermostats. The reactor tubes were each filled with approx. 1500 ml of the catalyst from Example 1. The menthol isomer mixture used can be found in the table below and was conveyed continuously into the tubular reactor by means of a membrane pump. The reactant was conducted through the tubular reactors either from the top (trickle phase) or from the bottom (liquid phase). Hydrogen was added by saturating the reactant at 0.6 to 1.2 MPa.

### Results

TABLE 1
Starting composition: 29.0% NM, 2.3% NIM, 55.7% M, 12.3% IM

| | | Product analysis | | | | | |
|---|---|---|---|---|---|---|---|
| T | Input | | | | | Sum of | |
| [° C.] | V [g/h] | NM ? | NIM | M | IM | menthols | M/IM* |
| 101 | 97 | 27.266 | 0.871 | 63.296 | 7.990 | 99.423 | 7.9 |
| 101 | 103 | 28.696 | 0.91 | 62.129 | 7.627 | 99.363 | 8.1 |
| 101 | 103 | 28.626 | 0.922 | 62.002 | 7.772 | 99.322 | 8.0 |
| 101 | 157 | 28.914 | 1.14 | 60.293 | 9.144 | 99.49 | 6.6 |
| 101 | 157 | 28.677 | 1.178 | 60.156 | 9.516 | 99.526 | 6.3 |
| 101 | 150 | 28.969 | 1.164 | 60.123 | 9.267 | 99.522 | 6.5 |
| 101 | 200 | 28.578 | 1.449 | 58.187 | 11.207 | 99.42 | 5.2 |
| 101 | 199 | 28.527 | 1.456 | 58.145 | 11.318 | 99.447 | 5.1 |
| 105 | 199 | 29.220 | 1.295 | 59.065 | 9.815 | 99.305 | 6.0 |
| 110 | 209 | 29.768 | 1.176 | 59.631 | 8.631 | 99.228 | 6.9 |
| 115 | 205 | 30.015 | 1.132 | 59.720 | 8.119 | 98.987 | 7.4 |
| 115 | 207 | 29.947 | 1.130 | 59.835 | 8.151 | 99.063 | 7.3 |
| 120 | 214 | 30.319 | 1.158 | 59.252 | 8.061 | 97.632 | 7.4 |
| 120 | 206 | 30.064 | 1.155 | 59.416 | 8.068 | 98.704 | 7.4 |
| 130 | 201 | 29.593 | 1.231 | 58.206 | 8.339 | 97.369 | 7.0 |
| 130 | 199 | 29.888 | 1.232 | 58.007 | 8.234 | 97.361 | 7.0 |
| 120 | 248 | 29.528 | 1.133 | 60.006 | 8.254 | 98.920 | 7.3 |
| 120 | 249 | 29.517 | 1.132 | 60.010 | 8.261 | 97.787 | 7.3 |

TABLE 2
Starting composition: 85.5% NM,
3.85% NIM, 6.65% M, 12.3% 0.97 IM

| | | Product analysis | | | | | |
|---|---|---|---|---|---|---|---|
| T | Input | | | | | Sum of | |
| [° C.] | V [g/h] | NM | NIM | M | IM | menthols | M/IM* |
| 115 | 353 | 30.172 | 1.001 | 60.895 | 7.266 | 99.335 | 8.4 |
| 115 | 357 | 30.129 | 0.991 | 61.008 | 7.245 | 99.373 | 8.4 |
| 125 | 352 | 29.856 | 1.180 | 59.633 | 8.209 | 98.698 | 7.3 |
| 114 | 349 | 30.325 | 0.979 | 60.962 | 7.078 | 99.344 | 8.6 |
| 114 | 356 | 30.281 | 0.962 | 61.135 | 6.988 | 98.404 | 8.7 |
| 114 | 355 | 29.639 | 0.934 | 61.688 | 7.111 | 99.372 | 8.7 |
| 111 | 353 | 29.317 | 0.847 | 62.655 | 6.679 | 99.498 | 9.4 |
| 111 | 352 | 29.194 | 0.834 | 62.764 | 6.638 | 99.430 | 9.5 |
| 111 | 362 | 31.777 | 0.897 | 60.655 | 6.069 | 99.399 | 10.0 |
| 111 | 357 | 30.270 | 0.815 | 62.305 | 6.097 | 99.487 | 10.2 |
| 109 | 349 | 30.057 | 0.801 | 62.537 | 6.119 | 99.513 | 10.2 |
| 109 | 351 | 30.119 | 0.806 | 62.445 | 6.135 | 99.505 | 10.2 |
| 106 | 347 | 29.313 | 0.719 | 63.602 | 5.910 | 99.545 | 10.8 |
| 106 | 349 | 29.167 | 0.707 | 63.799 | 5.854 | 99.527 | 10.9 |
| 106 | 349 | 29.088 | 0.701 | 63.861 | 5.854 | 99.504 | 10.9 |
| 103 | 349 | 28.540 | 0.622 | 64.905 | 5.477 | 99.544 | 11.9 |
| 103 | 354 | 28.451 | 0.616 | 64.985 | 5.496 | 99.547 | 11.8 |
| 100 | 354 | 27.982 | 0.558 | 65.795 | 5.240 | 99.574 | 12.6 |
| 100 | 344 | 28.074 | 0.558 | 65.747 | 5.191 | 99.571 | 12.7 |
| 97 | 344 | 28.260 | 0.512 | 65.679 | 5.106 | 99.557 | 12.9 |
| 100 | 349 | 27.728 | 0.526 | 66.208 | 5.13 | 99.592 | 12.9 |
| 100 | 347 | 27.590 | 0.519 | 66.339 | 5.188 | 99.636 | 12.8 |
| 100 | 171 | 29.148 | 0.568 | 64.911 | 4.960 | 99.587 | 13.1 |
| 100 | 175 | 29.557 | 0.627 | 64.145 | 5.261 | 99.590 | 12.2 |
| 100 | 164 | 28.553 | 0.600 | 65.108 | 5.390 | 99.651 | 12.1 |
| 95 | 175 | 27.987 | 0.540 | 65.885 | 5.182 | 99.594 | 12.7 |
| 95 | 168 | 27.847 | 0.521 | 66.116 | 5.090 | 99.575 | 13.0 |

TABLE 3
Starting composition: 99.7% D-menthol

| | | Product analysis | | | | | |
|---|---|---|---|---|---|---|---|
| T | Input | | | | | Sum of | |
| [° C.] | V [g/h] | NM | NIM | M | IM | menthols | M/IM* |
| 94 | 173 | 15.352 | 0.020 | 84.261 | 0.204 | 99.837 | 413.0 |

NIM = d,l-neoisomenthol, NM = d,l-neomenthol, M = d,l-menthol, IM = d,l-isomenthol
M/IM* = menthol/isomenthol ratio

The mixture from Table 3 is subjected to a fractional distillation (approx. 160 theoretical plates, reflux ratio 40:1). At the top of the column, 99.1% (−)-neomenthol is obtained. This preparation route is to date the only known industrially practicable way of preparing (−)-neomenthol.

TABLE 4
Starting composition: 99.5% d,l-menthol

| | | Product analysis | | | | | |
|---|---|---|---|---|---|---|---|
| T | Input | | | | | Sum of | |
| [° C.] | V [g/h] | NM | NIM | M | IM | menthols | M/IM* |
| 90 | 175 | 27.328 | 0.068 | 71.696 | 0.688 | 99.780 | 104.2 |
| 90 | 179 | 26.604 | 0.062 | 72.517 | 0.627 | 99.810 | 115.7 |
| 90 | 179 | 26.443 | 0.050 | 72.798 | 0.536 | 99.827 | 135.8 |
| 90 | 178 | 25.922 | 0.048 | 73.333 | 0.524 | 99.828 | 139.9 |
| 90 | 179 | 26.623 | 0.048 | 72.656 | 0.506 | 99.834 | 143.6 |

TABLE 5
Starting composition: 99.5% l-menthol

| | | Product analysis | | | | | |
|---|---|---|---|---|---|---|---|
| T | Input | | | | | Sum of | |
| [° C.] | V [g/h] | NM | NIM | M | IM | menthols | M/IM* |
| 91 | 172 | 15.1 | 0.020 | 84.331 | 0.202 | 99.653 | 417.4 |

The mixture from Table 3 is subjected to a fractional distillation (approx. 160 theoretical plates, reflux ratio 40:1). At the top of the column, 99.3% (+)-neomenthol is obtained. This preparation route is the only known industrially practicable way of preparing (+)-neomenthol.

What is claimed is:

1. Catalyst comprising ruthenium applied to a support material, the support material being aluminium oxide, characterized in that
the catalyst comprises at least one alkaline earth metal alkoxylate or
is obtainable by reacting a catalyst comprising ruthenium applied to a support material, the support material being aluminium oxide, with at least one alkaline earth metal alkoxylate.

2. Catalyst according to claim 1, characterized in that the aluminium oxide used as the support material has a BET surface area of at least 100 m²/g.

3. Catalyst according to claim 1 or 2, characterized in that the ruthenium content is 0.1 to 35% by weight.

4. Catalyst according to any of claims 1 to 3, characterized in that the alkaline earth metal alkoxylates used are those of the formula (I)
(R—O)₂M (I)
in which
R in each case independently is a primary, secondary or tertiary, cyclic or acyclic, branched or unbranched C1 to C20-alkyl radical which may optionally be further substituted by aryl, C₁-C₄-alkoxy or C₆ to C₁₄-aryloxy and
M is calcium, strontium or barium.

5. Catalyst according to claim 4, characterized in that the alkaline earth metal alkoxylates used are those of the formula (I) in which
M is barium.

6. Catalyst according to any of claims 1 to 5, characterized in that the amount of the alkaline earth metal alkoxylate which is used for preparation of the inventive catalyst is selected such that the molar ratio of ruthenium to alkaline earth metal is 30:1 to 1:30.

7. Use of the catalysts according to any of claims 1 to 6 for isomerization of stereoisomers of menthol or mixtures of such stereoisomers.

8. Process for isomerizing stereoisomers of menthol or mixtures of such stereoisomers in the presence of a catalyst according to any of claims 1 to 6.

9. Process according to claim 8, characterized in that d,l-menthol is prepared by catalytic isomerization of stereoisomers of menthol or mixtures of these stereoisomers.

10. Process according to claim 8, characterized in that
(+)-menthol (D-menthol) is isomerized to (−)-neomenthol or
(−)-menthol (L-menthol) to (+)-neomenthol or
(+)-isomenthol to (−)-neoisomenthol or
(−)-isomenthol to (+)-neoisomenthol.

11. Process according to any of claims 8 to 10, characterized in that it is conducted continuously.

12. Process according to claim 10, characterized in that the catalyst hourly space velocity is 0.005 to 5 kg of starting material per litre of catalyst and hour [kg/l*h].

13. Process according to any of claims 8 to 10, characterized in that it is conducted at a total pressure of 25 hPa to 30 MPa.
